(19) United States
(12) Patent Application Publication
(10) Pub. No.: US 2013/0224308 A1
(43) Pub. Date: Aug. 29, 2013

(54) GEL COMPRISING REACTIVE OXIDANT RELEASE AGENT

(71) Applicants: Wim De Windt, Sint-Amandsberg (BE); Frédéric Lakaye, Sella Plage (FR); Jan Dick, Gavere (BE)

(72) Inventors: Wim De Windt, Sint-Amandsberg (BE); Frédéric Lakaye, Sella Plage (FR); Jan Dick, Gavere (BE)

(73) Assignee: BIOREM ENGINEERING SARL, Sella Plage (FR)

(21) Appl. No.: 13/883,820

(22) PCT Filed: Nov. 8, 2011
(86) PCT No.: PCT/EP11/69602

§ 371 Date: May 7, 2013

(30) Foreign Application Priority Data
Nov. 8, 2010 (EP) 10190388.8

(51) Int. Cl. A61K 47/02; C09K 17/02; C09K 17/48; C02F 1/72; C09K 17/06

(52) U.S. Cl. CPC A61K 47/02; C02F 1/72; C02F 1/722; C09K 17/06; C09K 17/48; C09K 17/02
USPC 424/616; 252/175; 252/186.43; 252/186.1; 252/186.25

(57) ABSTRACT

Gel comprising reactive oxidant release agent The invention provides a gel composition comprising 5% to 75% w/w of an inorganic gelling agent other than silica based gelling agents, 0.5-60% w/w, preferably 0.5 to 30% w/w, of an inorganic oxidant release agent and at least 20% w/w of water, and possibly a stabilizer. It further provides a process for making same, comprising preparing a first solution of a dibasic phosphate salt in water comprising dissolved oxidant release agent, preparing a second and solution of a alkaline earth metal salt in water comprising dissolved oxidant release agent, and mixing both, first and second solutions, in a ratio of 1 to 1 to 10 to 1, followed by a concentration step. The gel composition of the invention is suitable for soil, sediment groundwater or water remediation purposes and disinfection or wound healing purposes.

### FIELD OF THE INVENTION

The present invention relates to the field of ground water remediation, soil or sediment remediation, water treatment, wound care and disinfection.

### BACKGROUND TO THE INVENTION

In environmental remediation processes, the supply of additional oxygen to contaminated soil and sediments is often used to stimulate the natural aerobic microbial breakdown of contaminants. However, oxygen on itself can also react directly with the pollutants by chemical oxidation. More suitable for this chemical treatment, are stronger oxidizers like hydrogen peroxide, calcium peroxide or persulfate.

Reactive oxidant release agents, such as IXPER 75C (Solvay) or ORC (Regenesis Inc.) are often used in contaminated soil or sediment remediation for their capacity to increase the dissolved oxygen concentration in water and groundwater and as such stimulate aerobic processes, such as e.g. microbial processes. They can also directly degrade contaminants by chemical oxidation. In order to convey slow release properties to these release agents, they can be chemically reacted into powdered precipitates, which slowly react with water over time and thus slowly release oxidant in this process.

Existing slow-release agents however have a number of drawbacks, which limit their applicability in practice. When these agents, for example in the form of calcium or magnesium peroxide, react with water, calcium or magnesium hydroxide is formed, generating a hard solid precipitate. The application of these components into aquifers or submerged sediments is not straightforward, and injection of water-based slurries of these compounds generate clogging problems. Moreover, application of reactive oxidant release agents as a fine powder generates fine particle dust which are irritating to skin and eyes. When hydrogen peroxide solutions are used as oxidant release agent, another problem arises as the injection hereof induces an immediate oxidation reaction of the close environment that cannot be controlled and that can be rather exothermic at higher peroxide concentrations, thus generating vapors and potentially causing volatilization of certain contaminants.

It is thus desirable to provide an improved formulation of oxidants or oxidant release agents for applications in environmental remediation. Such formulation should combine the capacity of oxidant slow-release over time (in contact with water), absence of precipitating reaction products, and user-friendly application.

US2002/0187007 (Schindler) discloses a method for remediating a contaminated region of a subterranean body of groundwater comprising the injection of substantially pure oxygen or oxygen in liquid form to naturally reduce the contaminants in the groundwater.

U.S. Pat. No.6,193,776 (Doetsch et al.) discloses a stabilizer (e.g. water glass) for inorganic peroxygen compounds to obtain a homogeneous calcium/magnesium peroxide, having a magnesium content of 4.2% to 17% by weight, a calcium content of 30 to 53% by weight, and an active oxygen content of 13 to 18% by weight.

US2003/0114334 (Coccia) claims to stabilize liquid compositions containing peroxides by thickening the liquids with water-soluble or water dispersible polymers.

US2008/0274206 (Lekhram et al.) discloses a stabilized liquid oxygen releasing composition comprising unspecified oxygen donor stabilizing agents and liquid binders. Stannates are often used as stabilizers for hydrogen peroxide, mostly in combination with additional stabilizers and/or chelating agents (see U.S. Pat. No. 7,169,237, Wang et al.) There are also different commercial phosphonates available to stabilize hydrogen peroxide and persulfate. For hydrogen peroxide, also1,10-phenanthroline, 8-hydroxyquinoline, citric acid, nitrilotriacetic acid, and ethylenediaminetetraacetic acid have a stabilizing effect (see U.S. Pat. No. 4,981,662, Dougherty, and U.S. Pat. No. 7,632,523, Ramirez et al.).

As the released oxidant is consumed by reaction with surrounding organic matter, an inorganic matrix is preferable over an organic matrix to deliver the oxidizing compounds. However, known peroxide-based gels are mostly based on organic substances like glycerin and propylene glycol (see U.S. Pat. No. 5,698,182, Prencipe et al.), polyvinylpyrrolidones (U.S. Pat. No. 5,945,032, Breitenbach et al.), polyacrylic acid thickening agents (see

US2004/0079920, Chadwick et al.), olefinically unsaturated carboxylic acid, and acrylate and methacrylate esters (see U.S. Pat. No. 7,045,493, Wang et al.).

Known inorganic gelling agents include fumed silica (U.S. Pat. No. 4,839,157, Mei-King Ng et al.) and Laponite, a synthetic clay (see US2007/0253918, Campanale et al.). Gels comprising such inorganic gelling agents are proposed in dental applications.

US2005/0011830 discloses a remediation formulation comprising 1-50% by weight of an oxidizing agent, 0.01-50% by weight of an inorganic thickening agent, 1-35% by weight inorganic salts and 1-90% by weight of a diluent. The oxidizing agent is selected from hydrogen peroxide, sodium or potassium permanganate, sodium persulfate, calcium hydroxide and magnesium hydroxide. The thickening agent is a silica based material preferably silica fume. The document does not disclose any exemplary formulation but in the case of bio-remediation, the composition may be complemented with nitrogen, potash, phosphate, microbes and biostimulants. All that can be found with respect to the viscosity of such compositions is that it should be similar to light oil (10-40 centistokes see [0027]) or in the range of 10 to 100 centistokes.

FR-2656949 discloses a decontaminating gel consisting in a colloidal solution comprising 8 to 25% by weight of an inorganic gelling agent, 3 to 10 mol/l of an inorganic base or acid, and 0.1 to 1 mol/l of an oxidizing agent having a normal oxido-reduction potential beyond 1400 mV/ENH (normal hydrogen electrode) in strong acidic medium, or of the reduced form of such oxidizing agent. When an inorganic acid is used, same is selected from hydrochloric acid, nitric acid, sulfuric acid and phosphoric acid; the selected gelling agent then is silica based. When an inorganic base is used , same is selected from soda or potash; the selected gelling agent then is based on Al2O03. The oxidizing agent is selected from Ce^{IV}, Co^{III} and Ag^{II}. It is advised that one may also use the reduced form of the relevant oxidizing agent but then in association with an oxidizer such a persulfate that is capable of oxidizing said reduced form back to the higher stage such compositions are used to decontaminate radioactive metal surfaces.

### PURPOSE OF THE INVENTION

The aim of the present invention is to overcome the drawbacks of currently existing oxidant release agents for applications in soil treatment, water treatment, sediment treatment, and disinfection such as: (1) clogging of tubing, solidification in injection filters etc. due to precipitation of reaction products at undesirable places (e.g. calcium hydroxide in the case of calcium peroxide), (2) immediate and strongly exothermic reactions upon contact with the organic material in soil and sediments, creating potentially toxic and dangerous fumes, foam and vapors, (3) limited reactivity of the applied oxidant release agents over time, (4) strong increase or decrease of pH in the groundwater or water phase caused by the oxidant release agent, (5) unwanted mobility issues resulting in the presence of the oxidant in places where there is no contaminant and vice versa,(6) the handling inconvenience of fine dust formation and respiratory irritation, and (7) the risk of reaction between organic gelling agent and oxidant or oxidant release agent. More specifically, this invention seeks to provide an improved composition for controlled-release of oxidants, more particularly magnesium peroxide and calcium peroxide. Both of these peroxides tend to be covered by an unreactive coating of transition metal hydroxide around the reactive oxidant-producing powder grain in contact with water. It is hence a purpose of this invention to overcome this auto-inhibition of the prolonged oxidant release.

The present invention further seeks to provide a controlled slow oxidant release system for reactive and unstable agents like hydrogen peroxide and persulfate that can readily be injected and pumped.

Briefly, the aim is to provide a soil remediation product that should be a stable, injectable and pumpable source of reactive oxidant when in contact with water, under adjustable kinetics.

Many of the current remediation products for contaminated soil, sediments and (ground) water are powders or liquids. These are rather mobile compounds which are easily washed out of the initial spot of injection with the moving water or groundwater. It has been found that a gel-like product comprising the oxidant release agent and being chemically inert to the oxidant and therefore not reacting with peroxides or other strong oxidants would solve this problem by keeping the oxidant release agent in its undiluted form and keeping it at the place where it is required.

### BRIEF DESCRIPTION OF THE INVENTION

According to a first aspect, the present invention thus concerns a reactive oxidant release agent, incorporated into an inorganic viscous gel so as to ensure a slow and controlled release of oxidant. More specifically, it consists in a gel composition comprising:
5% to 75% w/w of an inorganic gelling agent other than silica based gelling agents,
0.5 to 60% w/w, preferably 0.5 to 30% w/w, of an inorganic oxidant release agent, and
at least 20% w/w of water,
   the inorganic gelling agent being selected such that its oxidation stage is essentially not increased by the inorganic oxidant release agent.

Under the term gel, it is herewith understood that the composition should show a viscosity of at least 150 cP. A content of gelling agent below 5% w/w leads to a composition that in most cases shows too low a viscosity. Beyond 75% gelling agent, the composition is too tough to be suitable for the uses according to the invention.

The inorganic gelling agent is advantageously selected from alkaline earth metal salts of phosphate, preferably calcium phosphate or magnesium phosphate.

As far as the oxidant or oxidant release agent is concerned, lower concentrations from 0.5 to 3 or 5% w/w may be suitable in disinfection and wound healing applications, while higher concentrations beyond 5% w/w may be more suitable for soil and sediment remediation and water or groundwater treatment.

The content of water essentially determines the viscosity of the gel. A minimum of water is required for a gel showing at least 150 cP viscosity. The person skilled in the art will control the water content of the gel such as to obtain a gel suitable for the applications as envisaged and complying with the aim of the invention that is the provision of a stable, injectable and pumpable gel that is capable of releasing oxidant when in contact with water.

The composition may further comprise 0.5 to 10% w/w, preferably 2 to 5% w/w, more preferably 3% w/w, of a stabilizing agent. Such stabilizing agents are known per se and may be selected from phosphonates, such as AMP (Amino-tris-(methylene-phosphonic acid), ATMP (Amino tris(methylene phosphonic acid)), EDTMP (ethylenediamine tetra(methylene phosphonic acid)), DTPMP (diethylenetriamine penta(methylene phosphonic acid)), HDTMP (hexamethylenediamine tetra(methylene phosphonic acid)), PBTC (Phosphonobutane-tricarboxylic acid), PMIDA (N-(phosphonomethyl)iminodiacetic acid), CEPA (2-carboxyethyl phosphonic acid), HPAA (2-Hydroxyphosphonocarboxylic acid), 1,10-phenanthroline, 8-hydroxyquinoline, citric acid, nitrilotriacetic acid, and ethylenediaminetetraacetic acid. These stabilizing agents suitably protect the oxidant release agent from decomposition or reduction by environmental conditions such as light or other factors.

The oxidant release agent is advantageously selected from hydrogen peroxide, sodium persulfate, potassium persulfate, sodium permanganate, potassium permanganate, calcium peroxide, carbamide peroxide, magnesium peroxide, sodium percarbonate, peracetate and sodium perborate.

According to a further aspect of the invention, there is provided herein a method for producing a gel composition as described above, comprising
preparing a first solution of a dibasic phosphate salt in water comprising dissolved oxidant release agent;
preparing a second solution of an alkaline earth metal salt in water comprising dissolved oxidant release agent,
mixing both, first and second solutions, in a molar ratio of alkaline earth metal to phosphate of 0.5 to 1 to 4 to 1, and
concentrating to form a gel as defined above.

The stabilizing agent may be added to either or both solutions. One may also add stabilizing agent or oxidant after gel formation. Preferably, the dibasic phosphate salt is disodium phosphate and the alkaline earth metal salt is selected from a calcium salt, preferably calcium chloride, or a customary magnesium salt.

According to a preferred embodiment of the invention, the gel is concentrated to 90 to 20% water, by a concentration step known per se. One may also drive the concentration further and dry up to obtain a powder suitable for certain applications.

The gels of the invention have shown to be particularly suitable for use in soil, sediment or (ground)water remediation, and the invention thus particularly relates to soil, sediment and (ground)water remediation products comprising a gel as described above.

Due the fact that the invention gel is of inorganic nature (except the stabilizer), it is inert to biodegradation and does not add extra oxygen demand upon injection. The incorporation of the oxidant release agent into the gel structure leads to a stable product with a slow-release of oxidant over time.

The concentrated gel has been found to be stable, that is suffering essentially no decomposition or deactivation of oxidant, during storage and handling for a suitable determined period of time, with a viscosity of at least 150 cP that is particularly suitable for pumping, mixing and injecting processes. The product becomes active once applied into e.g. aquifers, wet soil or sediment matrices, and starts to produce oxygen at a slow rate in contact with water. Catalysts such as ferrous iron, micron sized or nano sized particles of zero-valent metals or solid metal oxides/transition metal oxides can be combined with the product for a release of reactive oxidant species with a higher oxidation potential, such as hydroxyl radicals or sulphate radicals.

It has also been found that the composition of the invention shows particularly interesting non-Newtonian properties which render it particularly suitable for applications in soil remediation. Actually, the viscosity diminishes significantly when shear is being increased and increases again when shear is decreased. These advantageous properties allow easy transfer of the gel by pumping, while it recovers the viscosity found appropriate for application to the polluted medium.

According to yet another aspect of the invention, there is provided herewith a powder composition suitable for the applications disclosed herein and obtained as described here above. In certain applications, powder compositions may be desirable despite some of the disadvantages same may entail. Such a powder composition according to the invention comprises an alkaline earth metal salt of phosphate with a molar ratio of alkaline earth metal ions to phosphate ions of 0.5 to 1 to 4 to 1, and an oxidant release agent in a weight ratio to the alkaline earth metal phosphate of 0.05 to 2.4.

The gels and powder compositions of the invention may also find suitable applications in the area of disinfection and wound care. More specifically, the invention provides a wound care product, such as a cream or ointment composition comprising a gel or powder as defined herein. A wound care product may also include a wound dressing bandage imbibed with a gel of the invention or including a powder of the invention.

### DETAILED DESCRIPTION OF THE INVENTION

The present invention provides a controlled release source of reactive oxidant species in the form of an inorganic viscous gel, for soil and sediment remediation. As mentioned before, the gel of the present invention comprises
5% to 75% w/w of an inorganic gelling agent other than silica based gelling agents,
0.5 to 60% w/w, preferably 1 to 30% w/w, of an inorganic oxidant release agent, and
at least 20% w/w of water.

According to a preferred embodiment of the invention, the gelling agent may be present in an amount of 10 to 55% w/w, According to a more preferred embodiment, the gelling agent is present in an amount of 10 to 40% w/w, or even more preferably 10 to 30% w/w.

The inorganic gelling agent is advantageously selected from alkaline earth metal salts of phosphate, preferably is calcium phosphate or magnesium phosphate. Particularly good results in soil remediation applications have been achieved with 20 to 25% w/w calcium phosphate gelling agent in the gel composition.

The oxidant release agent is advantageously selected from hydrogen peroxide, sodium persulfate, potassium persulfate, sodium permanganate, potassium permanganate, calcium peroxide, carbamide peroxide, magnesium peroxide, sodium percarbonate, peracetate and sodium perborate. According to a preferred embodiment, the inorganic oxidant or oxidant release agent is present in an amount of 0.5 to 45% w/w, more preferably from 0.5 to 30% w/w. Advantageously, the oxidant release agent is present in a ratio to the alkaline earth metal phosphate of 0.3 to 2.4.

The invention provides the combination of alkaline earth metal ions, such as calcium or magnesium ions, and phosphate ions present in two separate solutions, in a molar ratio of alkaline earth metal to phosphate of 0.5 to 1 to 4 to 1. Both ions are dissolved into a concentrated aqueous solution of an oxidant, such as an aqueous hydrogen peroxide solution. Both solutions are mixed at a certain ratio and concentration, until a viscous structure is obtained. If calcium ions are desired, these can be delivered from calcium chloride by dissolution into an aqueous solution of oxidant, e.g. an aqueous solution of hydrogen peroxide; whereas the phosphate can be delivered from disodium phosphate by dissolution into an aqueous solution of oxidant, e.g. an aqueous solution of hydrogen peroxide. After mixing both solutions, a gel is obtained after a concentration step, such as filtering the resulting liquid over a cellulose filter or other means of solid-liquid separation. The gelling agent may be present in an amount ranging from 5 to 75%. The resulting gel is inorganic and easily dispersible in water. The oxidant release agent utilized in the gel is present in an amount ranging from 0.5 to 60%, more preferably 0.5 to 30%, most preferably 13.75%. The oxidant release agents may be selected from or may be any combination of: hydrogen peroxide, sodium persulfate, calcium peroxide, carbamide peroxide, magnesium peroxide, sodium permanganate, potassium permanganate, sodium percarbonate, peracetate and sodium perborate. The stabilizing agent, that can be added to the gel for prolonged storage and stability, is present in an amount from 0.5 to 10%, more preferably 2 to 5%, most preferably 3% and may be selected from phosphonates, such as AMP (Amino-tris-(methylene-phosphonic acid), ATMP (Amino tris(methylene phosphonic acid)), EDTMP (ethylenediamine tetra(methylene phosphonic acid)), DTPMP (diethylenetriamine penta(methylene phosphonic acid)), HDTMP (hexamethylenediamine tetra(methylene phosphonic acid)), PBTC (Phosphonobutane-tricarboxylic acid), PMIDA (N-(phosphonomethyl)iminodiacetic acid), CEPA (2-carboxyethyl phosphonic acid), HPAA (2-Hydroxyphosphonocarboxylic acid), 1,10-phenanthroline, 8-hydroxyquinoline, citric acid, nitrilotriacetic acid, and ethylenediaminetetraacetic acid.

The combination of the above mentioned elements provides a viscous gel with a high concentration of oxidant release agent. Substantially no free oxidant (e.g. peroxide or persulphate) is measured in the gel, indicating an incorporation of the reactive oxygen species into the inorganic gel matrix (e.g. Ca₃(PO₄)₂·xH₂O₂).

Whenever a oxidant release agent is in the powdered form and is soluble in a gel matrix, the oxidant release agent can be mixed with the viscous inorganic gel for applications where additional, instant oxidative power is needed.

The slow release gel composition of the invention can be applied in soil or sediment by high pressure injection, at slightly elevated pressure or by percolation at atmospheric pressure. The viscosity of the gel can be modified by controlling the dewatering step at the end of the production process, e.g. by exposing the gel to increased temperature after production (e.g. 60 ° C. during 5 to 10 hours). The gel can be further dried (e.g. in an air flow) to an oxidant releasing powder.

As described above such powders may find suitable applications in soil and sediment remediation in certain cases and may still be preferred to gel compositions, despite certain disadvantages they may have compared to gel compositions. The powder compositions according to the invention comprises an alkaline earth metal salt of phosphate with a molar ratio of alkaline earth metal ions to phosphate ions of 0.5 to 1 to 4 to 1, and an oxidant release agent in a weight ratio to the alkaline earth metal phosphate of 0.05 to 2.4.

The gel compositions of the invention as well as the powder compositions of the invention may also be used for disinfection and wound care. They may be used in wound care ointments or wound dressing bandages.

The present invention is not restricted to the exemplified embodiments and the scope of protection extends to variations and modifications that fall within the scope of the claims.

### Example I

Preparation of an Inorganic Viscous Gel with Hydrogen Peroxide as Oxidant Release Agent

50g Na₂HPO₄·7H₂O was dissolved in 500 ml 27.5% hydrogen peroxide by magnetic stirring (ca. 1000 rpm) and gentle heating (ca. 50° C.) for approximately 15 minutes. Meanwhile, 500 ml 27.5% hydrogen peroxide was added to 16 g anhydrous calcium chloride that dissolved immediately without further manipulation needed. When the phosphate-mixture was completely dissolved, the two clear solutions were poured together and a thicker white substance started to form immediately. To remove the excess of liquid, this mixture was put over a paper filter with a pore size of maximum 5 μm. The remaining gel on the filter was further dried to the air for a few days. This finally yielded 140 g gel at pH 6.4. With these results, the oxygen capacity of the gel can be calculated to be 11.4% (w/w), i.e. the amount of oxygen that can be released from this formulation.

| Total mixed | | H₂O₂ | | Gelling | Water remaining |
|---|---|---|---|---|---|
| Volumes | Gel obtained | capacity | pH | Agent | in Gel |
| 1 L | 140 g | 22.9% | 6.4 | 16.8% | 60.3% |

### Example II

Preparation of an Inorganic Viscous Gel with a Stabilizing Agent and Hydrogen Peroxide as Oxidant Release Agent

The gel was produced according to Example I, with the difference that a phosphonate stabilizer (diethylenetriamine penta(methylene phosphonic acid)) was added to the Na₂HPO₄ solution at a concentration of 6% (v/v), just prior to addition of the CaCl₂ solution. After mixing the two solutions, it was left to settle for about one hour before it was put over a filter. The obtained gel had a total weight of 200 g, showed a pH 4.4 and an oxygen capacity of 12% (w/w). The texture was smooth and the gel did not release oxygen after 14 days at 20° C.

| Total mixed | | H₂O₂ | | Gelling | Water remaining |
|---|---|---|---|---|---|
| Volumes | Gel obtained | capacity | pH | Agent | in Gel |
| 1 L | 200 g | 24.3% | 4.4 | 11.8% | 63.9% |

### Example III

Preparation of an Inorganic Viscous Gel with Potassium Persulfate as Oxidant Release Agent

The gel was produced according to Example I, but the Na₂HPO₄ and CaCl₂ powders were dissolved in a 50 g/L K₂S₂O₈ solution instead of hydrogen peroxide. After filtration, 320 g of gel was obtained at pH 5.8 and the calculated sulfate radical capacity was 4.6% (w/w). The gel had a dry, granular appearance.

| Total mixed | | SO4 | | Gelling | Water remaining |
|---|---|---|---|---|---|
| Volumes | Gel obtained | capacity | pH | Agent | in Gel |
| 1 L | 320 g | 3.6% | 5.8 | 10.9% | 85.5% |

### Example IV

Preparation of an Inorganic Viscous Gel with a Stabilizing Agent and Potassium Persulfate as Oxidant Release Agent

The gel was produced according to Example III, but in order to stabilize the persulfate, a phosphonate (diethylenetriamine penta(methylene phosphonic acid)) was added to the Na₂HPO₄ solution at a concentration of 6% (v/v) prior to adding the CaCl₂ solution. After mixing the two solutions, it was left to settle for about one hour before it was put over a filter. The obtained gel was smooth and particularly suitable for injection in contaminated sites.

| Total mixed | | SO4 | | Gelling | Water remaining |
|---|---|---|---|---|---|
| Volumes | Gel obtained | capacity | pH | Agent | in Gel |
| 1 L | 270 g | 4.2% | 4.1 | 12.9% | 82.9% |

### Example V

Preparation of an Inorganic Viscous Gel with Hydrogen Peroxide as Oxidant Release Agent and Magnesium as Earth Alkali Metal

50 g Na₂HPO₄·7H₂O was dissolved in 500 ml 27.5% hydrogen peroxide by magnetic stirring (ca. 1000 rpm) and gentle heating (ca. 50° C.) for approximately 15 minutes. Meanwhile, 500 ml 27.5% hydrogen peroxide was added to 16 g anhydrous magnesium sulphate and stirred until a solution was obtained. When the phosphate-mixture was completely dissolved, the two clear solutions were poured together and a thicker white substance started to form slowly over 12 h. To remove the excess of liquid, this mixture was put over a paper filter with a pore size of maximum 5 μm after 12 h. This yielded 140 g gel at pH 6.1. With these results, the oxygen capacity of the gel can be calculated to be 12.3% (w/w), i.e. the amount of oxygen that can be released from this formulation.

| Total mixed | | H₂O₂ | | Gelling | Water remained |
|---|---|---|---|---|---|
| volumes | Gel obtained | capacity | pH | agent | in the gel |
| 1 L | 195 g | 24.6% | 6.1 | 10.5% | 64.9% |

### Example VI

### Oxidant Release Aspect of the Gels

The tests for oxidant release were executed in 750 mL closed receptacles. In the closed receptacles, 500 g of soil was wetted with 250 mL tap water. Hydrogen peroxide gel was obtained according to Example II and was dosed at a concentration of 20 mL/kg soil, while mixing it under the soil as a first test set-up. In a second test set-up, hydrogen peroxide gel was obtained according to Example II and was dosed at a concentration of 20 mL/kg soil,

by creating local reactive zones of 1 mL of oxidant releasing gel. In a third test set-up the slow release oxidant source calcium peroxide (powder) was used at a concentration of 12 g/kg. Hydrogen peroxide (27.5%) was used at a concentration of 20 mL/kg soil in a forth experiment. A fifth set-up was used as a control with no addition of oxidant releasing components. The oxidant release was measured by measuring the dissolved oxygen over time, as shown in Table 1.

During the measurements, the Dissolved Oxygen electrode (HANNA HI9828) was injected at the same coordinates every time and the oxygen concentration was measured 5 seconds after introducing the electrode at this spot.

TABLE 1
Oxygen concentration in water saturated soil samples as a
function of time, for different oxidant-release compounds

| | Calcium | Calcium | | | |
|---|---|---|---|---|---|
| | phosphate•hydrogen | phosphate•hydrogen | Calcium | Hydrogen | |
| Time | peroxide (ppm O₂) - | peroxide (ppm O₂) - | peroxide | peroxide | Control |
| (days) | mixed | oxygen zones | (ppm O₂) | (ppm O₂) | (ppm O₂) |
| 1 | 21.5 | 33.34 | 15.84 | 10.08 | 7.13 |
| 3 | 6.30 | 7.30 | 2.3 | 2.45 | 1.75 |
| 8 | 5.67 | 3.10 | 1.38 | 1.78 | 1.24 |
| 12 | 3.63 | 3.6 | 2.52 | 1.66 | 3.25 |
| 16 | 5.93 | 6.10 | 5.36 | 4.88 | 3.78 |

From the results in Table 1, it can be seen that an inorganic viscous gel comprising oxidant release agent(s) is a suitable source of continuous oxidant release over time. The following disadvantages were overcome by this invention:
No solidification: No formation of a precipitate that could solidify the matrix
No immediate exothermic reaction: The product has a slow-release capability because of encapsulation of the oxidant in a slow-release gel. Oxidant release is better controlled and can be varied by changing the concentration of oxidant during the production process.
No elevated pH: pH is close to neutral
No migration: the gel keeps the oxidant essentially in place
No dust formation during application

### Example VII

### Stability of the Gel

A total of 10 g gel, obtained from Example II, comprising reactive oxidative hydrogen peroxide was added to 1 L of demineralized water. The suspension was mixed and left standing during 7 days, in the dark at 20° C. After 7 days of incubation, the supernatant was analyzed for phosphate (according the procedure described in ISO 6874:2004). The water solubility of phosphate as measured is less than 0.06 mg PO₄³⁻/71.

The data shows that after 7 days of incubation, substantially no phosphate has been dissolved in water. This indicates a low water solubility of the gel in demineralized water and confirms the stability of the gel in water as well as the slow release properties

### Example VIII

### Thixotropic Properties of the Gel

### Materials and Methods

Three replicate samples of a gel produced according to the method described in Example II were analyzed. The dynamic viscosity was measured with a Brookfield DVII+Pro viscometer. During the dynamic viscosity measurement the shear rate was gradually increased, starting from 120 s⁻¹, increasing 5 s⁻¹ up to a final shear rate of 170 s⁻¹, subsequently the shear rate was gradually decreased up to a final shear rate of 120 s⁻¹. Measurements were performed at 19.6° C. The results are shown in Table 2 below.

Batch 1 consisted of a gel with a dry weight of 7.0%. Batch 2 consisted of a gel with a dry weight of 7.6%. Batch 3 consisted of a gel with a dry weight of 9.7%.

TABLE 2
Dynamic viscosity

| | batch 1 | batch 2 | batch3 |
|---|---|---|---|
| Shear | Viscosity | Viscosity | Viscosity |
| rate (s−1) | (kg · m⁻¹ · s⁻¹) | (kg · m¹ · s⁻¹) | (kg · m⁻¹ · s₋₁) |
| 120.00 | 18900.00 | 24400.00 | 161500.00 |
| 120.00 | 18300.00 | 23800.00 | 161000.00 |
| 120.00 | 18300.00 | 23700.00 | 139000.00 |
| 120.00 | 18300.00 | 23100.00 | 144500.00 |
| 120.00 | 18300.00 | 22400.00 | 125500.00 |
| 125.00 | 13450.00 | 15200.00 | 57250.00 |
| 130.00 | 9233.33 | 9533.33 | 43166.67 |
| 135.00 | 6950.00 | 7825.00 | 31750.00 |
| 140.00 | 5620.00 | 8020.00 | 21600.00 |
| 145.00 | 4666.67 | 5300.00 | 19583.33 |
| 150.00 | 3985.71 | 4185.71 | 20500.00 |
| 155.00 | 3450.00 | 3862.50 | 19937.50 |
| 160.00 | 3066.67 | 3344.44 | 18333.33 |
| 165.00 | 2740.00 | 2820.00 | 18050.00 |
| 170.00 | 2472.73 | 2490.91 | 20090.91 |
| 170.00 | 2409.09 | 2327.27 | 20227.27 |
| 170.00 | 2381.82 | 2154.55 | 20136.36 |
| 170.00 | 2363.64 | 2127.27 | 20227.27 |
| 170.00 | 2336.36 | 2109.09 | 20363.64 |
| 170.00 | 2309.09 | 2109.09 | 20954.55 |
| 165.00 | 2380.00 | 2310.00 | 22100.00 |
| 160.00 | 2588.89 | 2577.78 | 23666.67 |
| 155.00 | 2825.00 | 2637.50 | 23062.50 |
| 150.00 | 3157.14 | 2657.14 | 25785.71 |
| 145.00 | 3600.00 | 2950.00 | 28416.67 |
| 140.00 | 4040.00 | 3400.00 | 32300.00 |
| 135.00 | 4850.00 | 4200.00 | 36750.00 |
| 130.00 | 6100.00 | 5366.67 | 44333.33 |
| 125.00 | 8650.00 | 7450.00 | 61750.00 |
| 120.00 | 15000.00 | 14800.00 | 107000.00 |
| 120.00 | 15000.00 | 14600.00 | 112500.00 |
| 120.00 | 14900.00 | 14400.00 | 106500.00 |
| 120.00 | 14900.00 | 14400.00 | 113500.00 |
| 120.00 | 15300.00 | 14200.00 | 108500.00 |
| 120.00 | 15400.00 | 14500.00 | 121000.00 |

Batch 1: During the increase of shear rate a decrease of viscosity is measured: at a shear rate of 120 s⁻¹ a maximal viscosity of 18900 kg · m⁻¹ · s⁻¹ (1890.0 cP) was measured, at a higher shear rate of 170 s⁻¹ a minimal viscosity of 2309 kg · m⁻¹ · s⁻¹ (230.9 cP) was measured.
Batch 2: At a shear rate of 120 s⁻¹ a maximal viscosity of 24400 kg · m⁻¹ · s⁻¹ (2440.0 cP) was measured, a higher shear rate of 170 s⁻¹ resulted in a minimal viscosity of 2109 kg · m⁻¹ · s⁻¹ (210.9 cP).
Batch 3: At a shear rate of 120 s⁻¹ a maximal viscosity of 161500 kg · m⁻¹ · s⁻¹ (16150.0 cP) was measured, at a higher shear rate of 165 s⁻¹ a minimal viscosity of 18050 kg · m⁻¹ · s⁻¹ (1805.0 cP) was measured.

The dynamic viscosity of the gel was set to be between 2109 kg·m⁻¹·s⁻¹ and 18050 kg·m⁻¹·s⁻¹ at a shear rate of 170 s⁻¹ and to be between 18900 kg·m⁻¹·s⁻¹ and 161500 kg·m⁻¹·s⁻¹ at a shear rate of 120 s⁻¹ at a temperature between 19.5 and 19.6° C. The above data shows that the dynamic viscosity decreases with increasing shear rate. The gel of the invention thus is suitable for being pumped for transport in appropriate pipes, since it liquefies for the transport and retrieves its gel-like state thereafter.

### Example IX

### Field Test

### Site Description

A field study was carried out over an extended period of four months. A gel according to the invention, and produced according to Example II, was injected into a contaminated subsurface environment. A hydrocarbon leak contaminated the surrounding area downstream of the groundwater flow. The groundwater level was situated at 2 to 4 mbg (meter below ground level) and the lithological studies indicated sandy clay up to 4 mbg and compact brown clay from 4 to 7 mbg. Five infiltration wells were arranged upstream of the groundwater. Each infiltration well consisted of a PVC tubing of 7.5 m length and 80 mm inner diameter. A filter screen was installed in the PVC tubing with a mesh-size of 0.5 mm, over a length of 5-5.5 m. Monitoring wells were installed, 20-50 m downstream of the pollution source. During the tests the temperature of the groundwater was between 12.7 and 15.4° C.

### Application

A total of 200 L gel, produced according to Example II, was infiltrated into the subsurface by means of 5 infiltration wells. The infiltration was done under slight overpressure of 400 mbar by sealing a compressor onto the infiltration wells (to provide additional pressure). Every well was rinsed with 30 L of water after infiltration, which is a total of 150 L over the 5 wells.

### Results

The results in terms of average oxygen measurement over 5 monitoring wells are shown in Table 3. The samples were taken at regular intervals in a monitoring well located approx. 10 m from the nearest infiltration well. Samples were analyzed on-site for dissolved oxygen concentration. During sampling, oxygen was measured in a closed circuit flow cell, which is a sealed cell, connected with a sampling tube from the groundwater monitoring well to inhibit influence of oxygen from the atmosphere. The oxygen concentration value is taken after stabilization of the measured parameters. During the 12 weeks of testing, the dissolved oxygen concentration in the groundwater was found to be at or above oxygen saturation level (10-20 mg/L) in the infiltration wells.

TABLE 3
Dissolved Oxygen Concentration in monitoring wells

| | Dissolved oxygen |
|---|---|
| | concentration (average |
| Time after injection | over 5 monitoring wells) |
| At start | 2.6 mg/L |
| After 1 week | 11.1 mg/L |
| After 2 weeks | 9.6 mg/L |
| After 4 weeks | 13.8 mg/L |
| After 5 weeks | 11.0 mg/L |
| After 12 weeks | 12.8 mg/L |

After the infiltration of the gel, the dissolved oxygen concentration started to rise in the groundwater in the monitoring wells. This is taken as an indication of the slow release of hydrogen peroxide, the decomposition into water and oxygen and the transport of oxygen downstream to the monitoring wells (measurement after 1 week) by natural groundwater flow.

During the 12 weeks of testing, the pH in the groundwater remained in within the pH-range of 6.38 - 7.29 and the hydrocarbon odor disappeared in the headspace of the aquifer samples.

### Example X

### Accelerated Decomposition

### Materials and Methods

The determination of hydrogen peroxide in the gel was done using an adaptation from the method described by Schumb et al (1955). Since the hydrogen peroxide is slowly released from the gel, the gelling agent was solubilized to release the hydrogen peroxide.

This was accomplished by adding an acid (see below).

Hydrogen peroxide was quantitatively oxidized by titration with a potassium permanganate solution of known normality under acidic conditions.

### Reagents:

0.1 N Potassium permanganate (KMnO₄) solution
Nitric acid (65%)

Between 200-250 g of an oxidant-containing sample (gel or other) was heated to approximately 70° C. for 8 h in a 0.5 L beaker containing a lid to limit evaporation. The heating was achieved by placing the beaker on a heating plate and a magnetic stirrer was used to homogenize the product during heating. Sub-samples were taken every hour for hydrogen peroxide content analysis. A sub-sample of 250 mg was acidified with 500 μL HNO₃ (65%) to solubilize the product and acidify the solution. The surplus of nitric acid was used to maintain solution acidity. Nitric acid was preferred to sulfuric acid in order to prevent the precipitation of gypsum (CaSO₄) from the solution.

The potassium permanganate was titrated into the solution until the color changed into persistent light pink. At this end point the concentration of H₂O₂ is calculated by the formula:

$$\% H 2 O 2 = \frac{volume KMnO 4 \times 0 , 1 N \times 1 , 7005}{weight of the sample}$$

Where
volume KMnO₄ corresponds with the titrated volume
0.1 N corresponds with the 0.1 N potassium permanganate solution
1,7005 corresponds to the equivalent of each mL 0.1 N KMnO₄ to 1,7005 mg H₂O₂

### Gel of the Invention:

The gel comprising hydrogen peroxide obtained from Example II was tested according to the above mentioned procedure. Weight loss of the samples due to evaporation was measured and taken into account during the calculation.

### Hydrogen Peroxide—Control

A similar procedure was used to test the decomposition of hydrogen peroxide (27.5%) under these conditions. To stabilize the hydrogen peroxide an equivalent amount of phosphonic acid-based stabilizer as used in Example 2 was added (at approx. 2.7%).

### Fumed Silica Gelling Agent—Comparative Test

A fumed silica gelling agent (Carb-O-Sil) was added to a hydrogen peroxide solution (27.5%) at a concentration of 50g fumed silica per L hydrogen peroxide (27.5%). An equivalent amount of stabilizer based upon phosphonic acid compounds was added compared to the gel described in Example 2 above (approx. 2.7%).

### CaHPO₄ Gelling Agent

A CaHPO₄ gelling agent was added to a hydrogen peroxide solution (27.5%) at a concentration of 50 g CaHPO₄ per L hydrogen peroxide (27.5%). An equivalent amount of phosphonic acid based stabilizer was added compared to the gel of Example 2 above (approx. 2.7%).

### Results

The decomposition of the active hydrogen peroxide (oxidant) in the different tested compositions was measured by determination of the decrease of hydrogen peroxide over a period of 8 hours at 70° C. The resulting data is shown in Table 4, expressed as decrease of initial oxidant content over time. Weight loss of samples due to evaporation was measured, and taken into account during calculation.

TABLE 4
Decrease Of Hydrogen Peroxide Content

| | Gel of | CaHPO₄, | | |
|---|---|---|---|---|
| | Example II | powder | Fumed silica | H₂O₂ |
| Time | Decrease of | Decrease of | Decrease of | Decrease of |
| (h) | H₂O₂ (w/w %) | H₂O₂ (w/w %) | H₂O₂ (w/w %) | H₂O₂ (w/w %) |
| 0 | 0.00 | 0.00 | 0.00 | 0.00 |
| 1 | 3.51 | 5.11 | 12.73 | 9.94 |
| 2 | 5.21 | 8.16 | 11.59 | 13.14 |
| 3 | 3.98 | 8.91 | 14.78 | 25.92 |
| 4 | 8.25 | 5.27 | 14.27 | 20.15 |
| 5 | 10.15 | 9.24 | 15.68 | 21.06 |
| 6 | 9.92 | 7.76 | 15.64 | 20.59 |
| 7 | 9.68 | 7.66 | 15.96 | 19.53 |
| 8 | 9.34 | 7.44 | 19.93 | 20.56 |

### Conclusion

The amount (weight) of (phosphonate-stabilized) hydrogen peroxide in a H₂O₂ solution (27.5%) decreased by 20.56% compared to the original amount during an accelerated decomposition test (heating to 70° C. and maintaining this temperature during 8 hours under continuous stirring). This decrease of hydrogen peroxide during 8h incubation was used as a reference (control).

The hydrogen peroxide decomposition in the gel produced according to Example II was measured to be maximum 10.15% during a treatment of 8 h at 70° C. It is believed the structure of the gel interacts with the oxidant, effectively preventing its rapid decomposition over time at elevated temperature in this accelerated decomposition test. The combination of CaHPO₄ as gelling agent and hydrogen peroxide resulted in a decrease of hydrogen peroxide of less than 10% at 70° C. during 8 hours.

Hydrogen peroxide in combination with fumed silica as gelling agent resulted in a decrease of hydrogen peroxide of about 20% at 70° C., within 8 hours after heating started. The decomposition of hydrogen peroxide in the matrix of fumed silica was not significantly different from the control, and this decomposition was significantly higher than the decomposition of hydrogen peroxide in combination with a calcium phosphate based gelling agent. Without being bound by theory, it may be concluded that there are specific interactions between the oxidant and calcium phosphate-based gelling agents that allow the gel according to the invention to be a more stable source of oxidant release over time, when compared to other inorganic gel formulations of the prior art. More specifically, calcium-phosphate based inorganic gels containing oxidants allow better preservation of the oxidizing agent when compared to fumed-silica containing inorganic gels. This surprising observation leads the inventors to conclude that inorganic gels containing oxidant release agents according to this invention are a better, more stable, and thus a more prolonged source of oxidant release. This obviously is of utmost importance in soil remediation applications and others. In these applications, biochemical and chemical processes have relatively slow reaction kinetics (order of 6 months or more), and hence a slow source of continuous oxidant release is required.

### Example XI

### Gel Density

Three independent samples of 50 ml each were taken from the gel of Example 2 and from the gel of Example 3, and the weight was measured. Density was computed from these data. These tests were run in triplicate with samples from three independent batches of each formulation. Table 5 shows the measured weight of 50 mL gel and the calculated density thereof.

TABLE 5
Gel Density

| Gel comprising persulfate (EX 3) | | | Gel comprising H₂O2 (EX 2) | | |
|---|---|---|---|---|---|
| Volume | Weight | Density | Volume | Weight | Density |
| (mL) | (g) | (g/L) | (mL) | (g) | (g/L) |
| 50 | 54 | 1080 | 50 | 54.6 | 1092 |
| 50 | 55 | 1100 | 50 | 57 | 1140 |
| 50 | 57 | 1140 | 50 | 57.3 | 1146 |

The average density was calculated to be 1107±31 g/L for the gel of Example 3 and 1126±30 g/L for the gel of Example 2.

1. A gel composition comprising
5% to 75% w/w of an inorganic gelling agent other than silica based gelling agents,
0.5-60% w/w, , of an inorganic oxidant release agent and
at least 20% w/w of water the inorganic gelling agent being selected such that its oxidation stage is not substantially increased by the inorganic oxidant release agent.

2. The composition of claim 1, wherein the inorganic gelling agent is selected from alkaline earth metal salts of phosphate, including calcium phosphate or magnesium phosphate.

3. The composition of claim 1, wherein the inorganic oxidant release agent is present in an amount of 0.5 to 45% w/w.

4. The composition according to claim 1, wherein the oxidant release agent is selected from the group consisting of hydrogen peroxide, sodium persulfate, potassium persulfate, sodium permanganate, potassium permanganate, calcium peroxide, carbamide peroxide, magnesium peroxide, sodium percarbonate, peracetate and sodium perborate, or combinations thereof.

5. The composition according to claim 1, further comprising 0.5 to 10% w/w of a stabilizing agent.

6. The composition according to claim 5 wherein the stabilizing agent is a phosphonate selected from the group consisting of AMP (Amino-tris-(methylene-phosphonic acid), ATMP (Amino tris(methylene phosphonic acid)), EDTMP (ethylenediamine tetra(methylene phosphonic acid)), DTPMP (diethylenetriamine penta(methylene phosphonic acid)), HDTMP (hexamethylenediamine tetra(methylene phosphonic acid)), PBTC (Phosphonobutane-tricarboxylic acid), PMIDA (N(phosphonomethyl)iminodiacetic acid), CEPA (2-carboxyethyl phosphonic acid), HP AA (2-Hydroxyphosphonocarboxylic acid), 1,1 0-phenanthroline, 8-hydroxyquinoline, citric acid, nitrilotriacetic acid, and ethylenediaminetetraacetic acid.

7. The composition according to claim 1, wherein the viscosity is at least 1500*10⁻⁴ kg·m⁻¹·s⁻¹(150 cP).

8. The composition according to claim 1, further comprising a catalyst.

9. The composition of claim 8 wherein the catalyst is selected from the group consisting of ferrous iron, micron sized or nano sized particles of zero-valent metals or solid metal oxides or transition metal oxides.

10. The composition of claim 1, having a pH between 4 and 9.

11. A soil, sediment, water or ground water remediation product comprising a gel composition according to claim 1.

12. A disinfection or wound care product comprising a gel composition according to claim 1, wherein the oxidant is comprised between 0.5 and 5% w/w.

13. A method for producing a gel composition according to claim 1, comprising
preparing a first solution of a dibasic phosphate salt in water comprising dissolved oxidant release agent
preparing a second solution of an alkaline earth metal salt in water comprising dissolved oxidant release agent,
mixing both, first and second solutions, in a molar ratio of earth alkali metal to phosphate of 0. 5 to 1 to 4 to 1, and
concentrating to form a gel showing a viscosity of at least 150 cP.

14. The method according to claim 13 wherein a stabilizing agent is added to either one or both solutions.

15. The method according to claim 13 wherein a stabilizing agent is added after gel formation.

16. The method according to claim 13, wherein an oxidant is further added after gel formation.

17. The method according to claim 14, wherein the stabilizing agent is a phosphonate selected from the group consisting of AMP (Amino-tris-(methylene-phosphonic acid), ATMP (Amino tris(methylene phosphonic acid)), EDTMP (ethylenediamine tetra(methylene phosphonic acid)), DTPMP (diethylenetriamine penta(methylene phosphonic acid)), HDTMP (hexamethylenediamine tetra(methylene phosphonic acid)), PBTC (Phosphonobutane-tricarboxylic acid), PMIDA (N-(phosphonomethyl)iminodiacetic acid), CEPA (2-carboxyethyl phosphonic acid), HPAA (2-Hydroxyphosphonocarboxylic acid), 1,10-phenanthroline, 8-hydroxyquinoline, citric acid, nitrilotriacetic acid, and ethylenediaminetetraacetic acid.

18. The method according to claim 13, wherein the dibasic phosphate salt is disodium phosphate and the alkaline earth metal salt is a calcium salt or a magnesium salt, or calcium chloride.

19. The method according to claim 13, wherein the gel is dewatered to 90 to 20% water, by a concentration step.

20. The method according to claim 13, wherein the gel is further dewatered and dried to a powder.

21. A powder composition obtained by the process of claim 20 and comprising an alkaline earth metal salt of phosphate with a molar ratio of alkaline earth metal ions to phosphate ions of 0.5 to 1 to 4 to 1, and an oxidant release agent in a weight ratio to the alkaline earth metal phosphate of 0.05 to 2.4.

22. A disinfection or wound care product comprising a powder composition according to claim 21 wherein the oxidant is comprised between 0.5 and 5% w/w.

23. A method of producing a gel composition comprising 5% to 75% w/w of an inorganic gelling agent other than silica based gelling agents, 0.5-60% w/w of an inorganic oxidant release agent, and at least 20% w/w of water, wherein the inorganic gelling agent is selected such that its oxidation stage is not substantially increased by the inorganic oxidant release agent, the method comprising:
preparing a first solution of a dibasic phosphate salt in water comprising dissolved oxidant release agent,
preparing a second solution of an alkaline earth metal salt in water comprising dissolved oxidant release agent,
mixing both, first and second solutions, in a molar ratio of earth alkali metal to phosphate of 0. 5 to 1 to 4 to 1, and
concentrating to form a gel showing a viscosity of at least 150 cP.
